(19) United States
(12) Patent Application Publication
(10) Pub. No.: US 2024/0216221 A1
(43) Pub. Date: Jul. 4, 2024

(54) CHILLER FOR USE IN MEDICAL APPLICATIONS

(71) Applicant: Vivos, Inc., d/b/a Advanced Medical Isotope Corporation, Richland, WA (US)

(72) Inventors: James Duncan, Kennewick, WA (US); Christopher M. Smith, Richland, WA (US); Otto Stigge, Moscow, ID (US)

(73) Assignee: Vivos, Inc., d/b/a Advanced Medical Isotope Corporation, Richland, WA (US)

(21) Appl. No.: 18/401,421

(22) Filed: Dec. 30, 2023

Related U.S. Application Data
(60) Provisional application No. 63/436,562, filed on Dec. 31, 2022.

(51) Int. Cl. A61J 1/16; A61N 5/10; F25D 11/00; F25D 31/00

(52) U.S. Cl. CPC A61J 1/165; A61N 5/1001; F25D 11/003; F25D 31/006; A61N 2005/1021

(57) ABSTRACT

A chiller for maintaining medical materials at a temperature below ambient room temperature preferably includes a casing, and a thermoelectric cooler having a hot side and a cold side that is coupled to and supported by the casing. A fluid mover moves fluid into thermal contact with a hot side to remove heat therefrom. First and second vial receivers are in thermal contact with the cold side of the cooler for receiving first and second medical materials containing vials. A stirrer is provided for stirring the medical material in the first vial and/or the second vial.

This application claims benefit of priority to James Duncan et al. U.S. Provisional Patent application No. 63/436,562, which was filed on 31 Dec. 2022, and is fully incorporated herein by reference.

### I. TECHNICAL FIELD OF THE INVENTION

The present invention relates to chillers, and more particularly, to chillers for uses in the medical applications

### II. BACKGROUND OF THE INVENTION

It is not unusual for medical products to require storage for periods of time under temperature-controlled, refrigerated conditions, so that the product is kept at a temperature lower than the typical ambient temperature of the room in which the product is stored. Additionally, some products have a further need to be stirred during storage to keep the ingredients or materials of the product well distributed, and to prevent them from reverting to an undesirable separated or clotted state.

One such class of materials are implantable materials such as implantable radioactive particle suspensions. An example of such an implantable radioactive particle suspension is shown in Korenko et al. U.S. Pat. No. 11,478,557, which issued on Oct. 25, 2022, and which is incorporated fully herein by reference. The Korenko patent is owned by the applicant of the present invention.

Materials such as these often require special handling. Typically, the materials are prepared at the manufacturer's location and placed in vials and shipped, often by expedited shipping. to the location where they will be used, which is typically a hospital or clinic. At the hospital or clinic, the storage or shipping vials are ultimately forwarded to a treatment or operating room where the materials will be implanted into a patient. During the time that the implantable materials are in the treatment or operating room, the vials must be both chilled and stirred.

Before the implantable materials are implanted into the patient, they are transferred from the shipping vials into an application or implanting vial, such as a syringe which is used to apply the medication to the patient. With a syringe this application takes the form of injecting it into the patient. Preferably, this is accomplished either just before the procedure begins, or sometime early during the procedure, so that the implantable-material-filled syringes will be available when the physician decides to implant the materials.

For this reason, the applicants have found that it is helpful to provide a device that is capable of chilling and holding not only the storage/shipping vials, but also the application/implant vials such as the syringes. One object of the present invention is to provide such a device for holding vials and providing a chiller that is capable of both stirring the implantable medical material, and maintaining the implantable medical materials at a temperature below normal ambient temperature. In this regard, ambient room temperatures are generally considered to be in the range of 67ºF to 78° F.

### III. SUMMARY OF THE INVENTION

In accordance with the present invention, a chiller is provided for maintaining medical materials at a temperature below ambient room temperature.

The chiller preferably comprises a casing having an interior that is configured for receiving a plurality of components. A thermoelectric cooler is coupled to and supported by the casing. The thermoelectric cooler includes a hot side and a cold side. A fluid mover is provided for moving fluid into thermal contact with the hot side to remove heat from the hot side.

A first vial receiver is placed in thermal contact with the cold side of the thermoelectric cooler, and is configured for receiving a first vial having an interior configured for receiving medical materials. A second vial receiver is also provided that is in thermal contact with the cold side of the thermoelectric cooler. The second vial receiver is configured for receiving a second vial having an interior configured for receiving a medical material. A stirrer is provided for stirring the medical material in the first vial and/or the second vial.

In an exemplary embodiment, a first vial receiver comprises of at least two, first vial receivers, with the first vial receiver being configured for shipping and/or storage of the medical material. The first vial receiver can comprise a plastic container having a metal bottom for facilitating heat transfer between an exterior and an interior of the container, and the container can be comprised of a radiation attenuating material.

Additionally, the first vial can comprise at least two, first vials and the stirrer is configured for stirring medical materials in the first two vials. Additionally, at least one applicator can be provided that is configured for applying medical materials to a patient, and the chiller can include at least one applicator receiver in thermal contact with the thermoelectric cooler for maintaining medical materials in the applicator below ambient temperature.

Further, the applicator can comprise a body having an exterior surface, an interior surface for defining an interior needle engaging end, an open plunger receiving proximal end, and a plunger insertable into the interior. The exterior surface of the body includes an annular groove configured to be engaged by the user's fingers facilitating movement of the plunger relative to the body.

In a further preferred embodiment, the applicator can comprise a body having an exterior surface, an interior surface for defining a needle engaging distal end, an open, plunger receiving proximal end, and a plunger insertable in the interior. The interior surface of the body may include an annular groove configured to be engaged by the user's fingers for facilitating movement of the plunger relative to the body.

A locking mechanism can be provided that is engageable with the applicator and applicator receiver for fixedly positioning the applicator and the applicator receiver. The locking mechanism includes a slot formed at a proximal end of the applicator receiver, and thumb screw insertable in the slot engageable with the annular groove of the applicator.

The chiller can further comprise an applicator receiver that is coupled to the casing and itself comprises a tubular cavity having an open proximal end for receiving the applicator and a closed distal end. The proximal end of the tubular cavity is disposed at a higher position than the distal end to position an applicator placed therein at an oblique angle relative to the chiller testing of the resting surface.

The chiller can further include an applicator receiver and an applicator configured for applying medical materials to a patient. The applicator receiver can comprise at least one tubular passageway configured for receiving the applicator within the passageway.

Additionally, an applicator receiving radiation shield can be provided that has a hollow interior for interiorly receiving the applicator, and an applicator locking mechanism engageable between the radiation shield and the applicator for maintaining the applicator within the hollow interior of the radiation shield. The applicator can include an angular groove and the locking mechanism can comprise a threaded screw having an end receivable into the angular groove of the applicator.

The chiller may also include an applicator receiver that includes a thermally conductive support member that is coupled to the casing and is in thermal contact with the thermoelectric cooler. The support member may include at least two tubular passageways configured for receiving the tubular shaped applicators having a proximal and a distal end. The two tubular passageways include relatively raised proximal ends for receiving the proximal end of the syringe shaped applicators, and a distal end for receiving the distal end of the applicators, to position the applicators at an oblique angle to the resting surface upon which the chiller rests.

Preferably, the casing includes a first casing member for housing a fluid mover, a second casing member to which the thermoelectric cooler is mounted, and a third casing member to which an applicator receiver is coupled. The applicator receiver is in thermal contact with the thermoelectric cooler.

Most preferably, the casing includes a lower casing member, and further comprises a heat sink in thermal contact with the outside of the thermoelectric cooler, and wherein the casing includes apertures for allowing cooling fluid to pass between ambient atmosphere and the interior of the casing and heat sinks.

Preferably, the fluid mover comprises an air mover for drawing ambient air into the interior of the first casing member, moving the ambient air through the applicators and into an interior of the first casing member, moving the air past the heat sink to absorb heat from the heat sink and exhausting the heat from the heat sink from the casing through the apertures. A heat sink receiving chamber can also be provided for encasing the heat sink.

Alternately, a container can be provided for encasing a vial containing a radioactive material. The container is comprised of a radiation attenuating material having a bottom, a side wall including an exterior surface, an interior surface, an interior cavity having a top opening, and being defined by the interior surface, and a thickness defined by the distance between the interior surface and the exterior surface. A cap member is placeable over the top opening. The cap member has a top surface, a bottom surface, and a thickness defined by the distance between the top surface and the bottom surface.

The thickness of the cap member and the thickness of the side wall are sufficiently great so as to absorb sufficient radiation emitted by the radioactive materials, so that radioactivity levels outside the container are at acceptably safe levels.

Preferably, a locking mechanism is provided for maintaining a second container within the container. The container includes a metal bottom for facilitating cooling of the container, and the radioactive materials contained within the second container.

These and other features and advantages will be described in the attached Description of the Drawings and Description of the Invention presented below, but are only limited by the breath of the claims included herein.

### IV. BRIEF DESCRIPTION OF DRAWINGS

FIG. 1 is a perspective view of the chiller of the present invention, showing a partial disassembly of the casing, with the top member and middle member removed, to expose the thermoelectric (Peltier) coolers and the heat sink;

FIG. 1A is a further exploded view, similar to FIG. 3, showing one of the heat sinks removed, to expose the fan member of the air handling portion of the chiller;

FIG. 2 is a perspective schematic view of the chiller wherein some of the components are shown in transparency;

FIG. 2A is a perspective view of an exemplary thermos electric or Peltier cooler;

FIG. 3 is a front-biased perspective view of the present invention, showing some of the components in transparency;

FIG. 4 is a perspective view of the magnetic stirrer assembly of the present invention;

FIG. 4A is a front view of a portion of the venting ports of the air handler assembly;
7262 Fishers Crossing Dr, Fishers, IN 46038 FIG. 5 is another perspective left rear side biased view of the chiller, showing some components in transparency;

FIG. 6 is an enlarged version of a portion of the view shown in FIG. 8

FIG. 7 is an enlarged perspective view of a portion of the front of the chiller, showing the syringes inserted into the syringe receiving members, and a portion of the air handler assembly;

FIG. 8 is another perspective view showing a portion of the front and top of the chiller;

FIG. 9 is a bottom biased perspective view showing the upper assembly and a portion of the magnetic stir assembly;

FIG. 10 is a top partial view of the chiller, taken from the top, showing the vial receiving member, including the covering therefor;

FIG. 11 is a side view of the shielded syringe of the present invention;

FIG. 11A is a prior art shielded syringe wherein the elements of the prior art device include an “a” after the part number;

FIG. 12 is a perspective view of the syringe radiation shield of the present invention, showing a syringe coupled thereto;

FIG. 12A is a top view of the syringe shield of the present invention without a syringe coupled thereto;

FIG. 12B is a side view of the syringe shield of the present invention;

FIG. 12C is a sectional view of the syringe shield of the present invention;

FIGS. 13-24 are views of an alternate embodiment chiller of the present invention wherein:

FIG. 13 is a perspective view of the upper casing member of the alternate embodiment

FIG. 14 is a front biased isometric view of the upper casing member of the present invention;

FIG. 15 is a front isometric view of the alternate embodiment chiller showing the electrical power source plug;

FIG. 16 is a front perspective view of the present invention, shown in transparency;

FIG. 17 is another front perspective view of the chiller alternate embodiment shown in transparency;

FIG. 18 is a sectional view taken along lines 22-22 of FIG. 21;

FIG. 19 is a perspective rear view of the present invention alternate embodiment shown in solid;

FIG. 20 is another perspective rear view of the present invention, shown in solid;

FIG. 21 is a sectional view taken along lines 25-25 of FIG. 24;

FIG. 22 is a sectional view taken along lines 26-26 of FIG. 25;

FIG. 23 is a sectional view taken along lines 27-27 of FIG. 24;

FIG. 24 is a sectional view taken along lines 28-28 of FIG. 24;

FIG. 25 is a perspective side view of the syringe shield and vial shield of the present application;

FIG. 26 is a perspective view of the syringe shield of FIG. 14, with a syringe inserted therein;

FIG. 27 is a side perspective view of the vial shield of FIG. 29, depicted with the vial located therein;

FIG. 28 depicts an exemplary radiation attenuating shipping container according to a first form of the present application;

FIG. 29A-29G depicts an alternative radiation attenuating shipping container, according to a further form of the present application;

FIG. 30 depicts a radiation attenuating shipping container, as described with regard to FIG. 29-29G, in a fully assembled configuration;

FIG. 31 depicts the radiation attenuating shipping container of FIG. 28, with the lid removed therefrom in a ready to load configuration;

FIG. 32 depicts the radiation attenuating shipping container of FIG. 28, in a fully loaded and ready-to-ship configuration;

FIG. 33 is a perspective view of another alternate embodiment of the present invention;

FIG. 34 is another perspective view of the embodiment of FIG. 33;

FIG. 35 is a transparent perspective view of the upper raising of the embodiment of FIG. 33; and

FIG. 36 is a solid perspective view of the upper casing of FIG. 35.

### V. DETAILED DESCRIPTION

The description that follows describes, illustrates and exemplifies one or more particular embodiments of the present invention in accordance with its principles. This description is not provided to limit the invention to the embodiment or embodiments described herein, but rather to explain and teach the principles of the invention in such a way to enable one of ordinary skill in the art to understand these principles and, with that understanding, be able to apply them to practice not only the embodiment or embodiments described herein, but also other embodiments that may come to mind in accordance with these principles.

The scope of the present invention is intended to cover all such embodiments that may fall within the scope of the appended claims, either literally or under the doctrine of equivalents.

It should be noted that in the description and drawings, like or substantially similar elements may be labeled with the same reference numerals. However, sometimes these elements may be labeled with differing reference numbers, such as, for example, in cases where such labeling facilitates a clearer description. Additionally, the drawings set forth herein are not necessarily drawn to scale, and in some instances, proportions may have been exaggerated to more clearly depict certain features. Such labeling and drawing practices do not necessarily implicate an underlying substantive purpose.

Furthermore, certain views are side views which depict only one side of the device or one set of components of a multi set array of components. However, it will be understood that the opposite side and other component sets are usually identical thereto. The present specification is intended to be taken as a whole and interpreted in accordance with the principles of the present invention as taught herein and understood by one of ordinary skill in the art.

As best shown in the figures, the medical materials chiller 10 has a casing 12. The casing or housing 12 includes an upper casing member 16, a lower casing member 18, and an intermediate casing member 22. Preferably, each of the casing members 16, 18, 22 are constructed out of a plastic material of the type that can withstand antiseptic cleanings, such as by cleaning in an autoclave or with harsh chemicals.

The upper casing 16 and intermediate casing 22 define an upper interior chamber 26 that is generally hollow or alternately may be filled with a solid block of heat conductive material which has been formed to include hollow portions such as syringe receiving passageways. The lower casing member 18 and the intermediate casing member 22 define a lower interior chamber 28 that is also generally hollow, but includes a plurality of components therein.

An air handler assembly 32 is contained within the lower interior chamber 28, along with a magnetic stirrer 34. The lower casing member 18 is generally open-box shaped, and rectangularly cuboid in configuration. The lower casing member 18 includes a bottom panel 44, and four side panels 48, including first short side panel 50, second short side panel 52, first long side panel 54, and second long side panel 56. The four side panels 48 are disposed in a plane that is generally perpendicular to the plane of the bottom panel 44, and comprise the sides of the open-box shaped lower casing member 18.

A first active heat sink 68 is disposed in a first heat sink receiving chamber 67 and is in thermal contact with the “hot” electrode of the first thermoelectric cooler, which is often referred to as a Peltier cooler 78. The first heat sink receiving chamber 67 is sized and configured for receiving the first active heat sink 68, and includes a fan receiving shelf 70, on which the fan 72 of the first active heat sink 68 is disposed.

The first active heat sink 68 also includes a finned aluminum heat sink 74, having an enlarged diameter portion, and a reduced diameter central portion 76. The reduced diameter central portion 76 is provided for receiving a first Peltier cooler 78 in a thermally conductive relationship with the finned aluminum heat sink member 74. The thermoelectric cooler 78 is received on the top surface of the reduced diameter central platform portion 76.

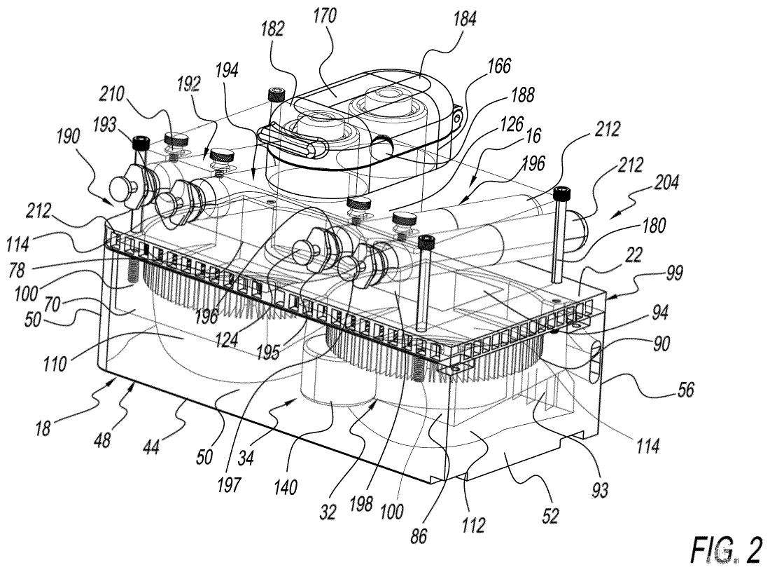

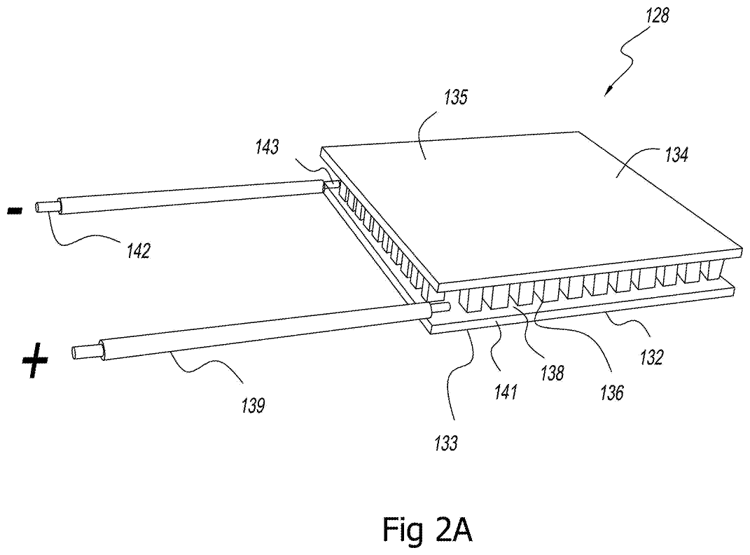

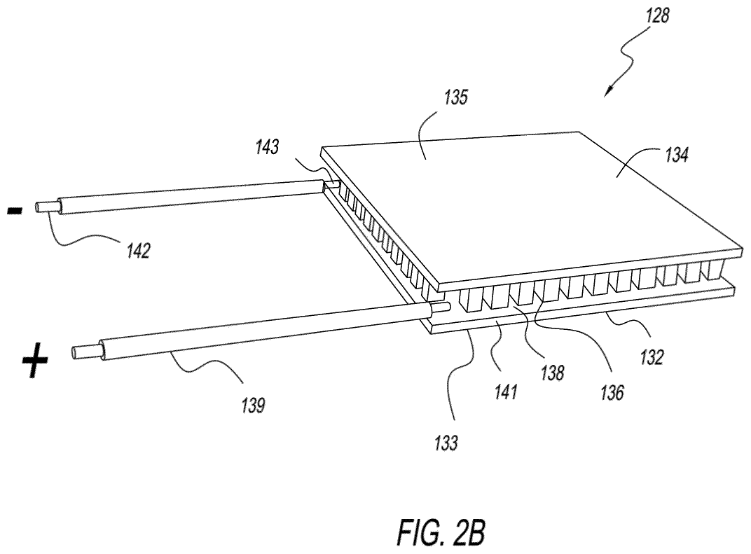

A view of a thermoelectric cooler 128 is shown in FIG. 2A. A thermoelectric cooler, which is often referred to as a Peltier cooler is a semi-conductor-based electronic component that works on the Peltier Effect. The Peltier Cooler 128 serves as a small refrigerator or heat pump, depending upon the application. When DC current flows through it 128, one side 134 becomes a cold side 134 and the other side 132 becomes a hot side 132. In order to continuously pump heat hot from the cold side 134 to the hot side 132 and keep it cooler, a heat sink is required at the side of the thermoelectric (or Peltier) cooler 128.

The components of a typical Peltier cooler 128 include a second, cold side ceramic or aluminum substrate 135 which is provided for absorbing heat from an item in thermal contact with the substrate 135. The cooler 128 also includes a first, hot side ceramic or aluminum substrate 133 that is provided for absorbing heat from the cold side substrate 135, and transferring that heat to a heat sink, such as heat sink 68 or heat sink 90.

A plurality of P type semi-conductors 136 and N type semi-conductor pellets 138 are disposed between and in contact with the first ceramic substrate 133 and second ceramic substrate 135, for providing the heating and cooling effect achieved by the thermoelectric cooler 128.

A first wire lead 139 is in electrical contact with the semi-conductors through a conducting to have a 141 that is preferably made from copper. A second wire lead 142 is electrically coupled to the device through a second conducting tear 143 for introducing the negative current to the device.

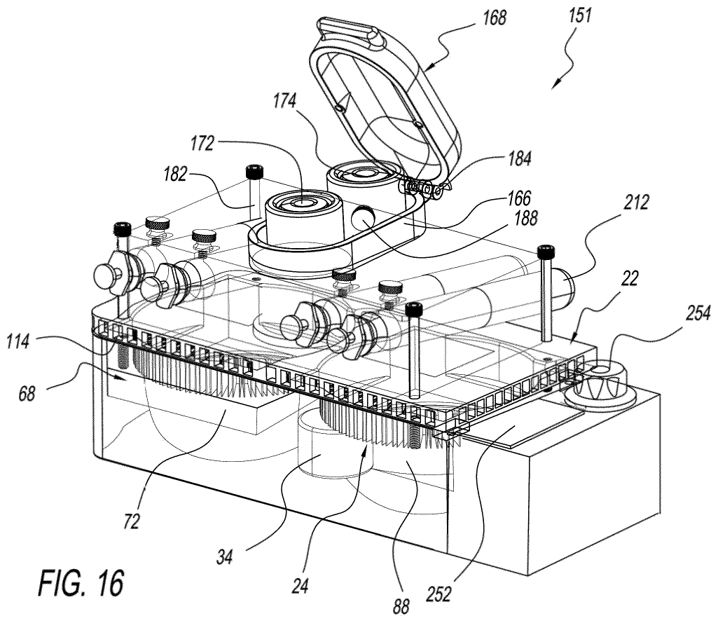

A second heat sink receiving chamber 82 is provided in the lower casing member 18, for receiving a second active heat sink 84. The second heat sink receiving chamber 82 includes a fan receiving shelf 86 for receiving the second fan 88 (FIG. 16), which is similar, if not identical to the first fan 72. The second active heat sink 84 is generally similar to the first active heat sink 68.

The second active heat sink 84 includes the second fan 88, and a finned aluminum heat sink 90, that includes an enlarged diameter portion, and a reduced diameter central platform portion 92. The underside of a second Peltier cooler 94 is in thermal conduct with, and disposed on the reduced diameter central portion 92.

The Peltier coolers 78, 94 have cold sides 134 and hot side 132. The cold sides 134 are the sides that face upwardly, and are positioned adjacent to the vials and syringes. The hot sides 132 are placed against the finned aluminum heat sinks. The Peltier coolers 78, 94 are reasonably efficient, and they have been able to cool the adjacent chamber of the device 10 to about 5° C. within about 15 minutes, by using two of 48-watt Peltier coolers 78, 94.

The Peltier coolers 78, 94 are also very effective at keeping the cooled material within a desired temperature range. Preferably, the Peltier coolers 78, 94 keep the material in a range of somewhere between about 2° C. and 8° C. The materials in the syringes should not be allowed to freeze by getting cooler than this. A small controller (not shown) is operably coupled to the Peltier coolers 78, 94, to control their operation, so that they maintain the appropriate temperature, and neither freeze the cooled medical materials, nor allow them to get too warm.

The second long side panel 56 of the lower casing unit 16 includes a plurality of intake apertures 93. The intake apertures 93 enable air to flow into the first 110 and second 112 intake chambers to cool the heat sinks 74, 84 and thereby cooling the Peltier coolers 78, 94. The first intake chamber 110 is disposed under the first heat sink 74 and Peltier cooler, and the second intake chamber 112 is disposed under the second heat sink 84 and Peltier cooler 94.

The lower casing member 18 includes an upper edge 96 and a transverse central member 97, that extends between the first long side panel 54 and second long side panel 56. The upper edge 96 and upper surface of the transverse central member 97 are sized and positioned to receive the lower edge 98 of the perimetral skirt of the intermediate casing member 22. Vertically oriented bolt receiving apertures 100 are threaded for receiving threaded bolts 180 that couple together the upper casing member, intermediate casing member 22 and lower casing member 18.

An output shaft receiving aperture 102 is disposed in the middle of the transverse central member 97, and is sized and positioned for receiving the output shaft 104 of the magnet member 34. The intermediate casing member 22 also includes a horizontally disposed panel 106. The perimetral skirt 99 includes a plurality of exhaust apertures 114, through which air that has already passed over the first and second heat sinks 74, 84 can be exhausted to the ambient atmosphere.

The intermediate member 22 also includes four bolt receiving apertures 113, that are aligned with the bolt receiving apertures 100 of the base member, and through which the connecting bolts 180 pass. The horizontally disposed panel 106 also includes first and second Peltier cooler receiving apertures 116, 118 for receiving the first and second Peltier coolers 78, 94 respectively.

A central recessed portion 120 is formed in the horizontally disposed panel 106, and is generally positioned at the center of the horizontally disposed panel 106. The horizontally disposed panel 106 of the intermediate casing member 22 includes a transverse member 119 that extends transversely between the first and second long sides, and is sized, configured and positioned to engage the transverse member 97 of the lower casing member 16.

The central recessed portion 120 is sized and positioned for receiving the magnet housing 122, along with the first magnet 124 and second magnet 126 of the magnet stirrer member 34. The central aperture 130 is formed in the central recessed portion 120 for receiving the output shaft 104 of the magnet member 34.

The magnet stirrer 34 has a motor 140, an output shaft 104, and a magnet 122, that includes first and second magnets 124, 126. Magnets of the type shown are commercially available from a wide variety of sources, and preferably, the magnet member 34 is designed to work off of a 12 V DC electrical input.

The upper casing member 18 includes a top panel 146, a first short side panel 148, a second short side panel 150, a first long side panel 152, and a second long side panel 154. As with the lower casing member 18, the upper casing member 16 is generally rectangularly cuboid in configuration.

A vial receiver 164 extends generally vertically at the center of the upper casing member 16. The vial receiver 164 includes a collar portion 166 which extends above the top surface of the top panel of the upper casing member 16. A cap 168 is hingedly coupled to the collar 166, and is movable between an open position and a closed position.

The vial receiver 164 includes a vial receiving chamber 170 into which a first vial 172, and a second vial 174 can be placed. The first and second vials 172, 174 contain the medical material that is being chilled by the chiller that can be placed and are the vials in which the medical material is placed for shipment to the hospital or facility, and/or is stored in prior to its placement into an application vial such as the syringes shown herein.

A coin shaped first vial receiving platform 176, is preferably made out of aluminum, and is placed on the base of the vial receiving chamber 170 to be positioned under the first vial 172. A coin shaped second vial receiving platform 178, is also made preferably from aluminum, and is positioned at the bottom of the vial receiving chamber 170, under the second vial 174.

The upper casing member 16 includes four bolt receiving passageways 179, for receiving four bolts 180, each of which has a threaded end 183. The bolts 180 pass through the receiving passageways 179, through mating apertures 113 in the intermediate casing member 22, and into blind holes 100 in the lower casing member 16, that are threaded to receive the threads 183 of the bolts 180.

When engaged in the passageway, 179 and when received by the threads of the base member 16, the bolts 180 securely engage and couple the three casing members 16, 18, 22 together. However, the bolts 180 also are removable from their threaded engagements with the base member 16 to permit the three casing members 16, 18, 22 to be separated from each other, which both facilitates manufacture and repair of the device 10

A cylindrical first vial shield 182 is placed over the first coin shaped vial plate 176, and is sized and configured for interiorly receiving the first vial 172 in the vial receiving chamber 170. Likewise, a cylindrical second vial shield 184 is disposed over the second coin shaped vial platform 178 and is sized and configured for receiving the second vial 174 in the vial receiving chamber 170. As will be discussed below, the vial platform 176 can be coupled with the vial shield 182 to serve as the base 176 of the vial shield 182.

The purpose of the first and second vial shields 182, 184 is to contain the radiation within the vials 172, 174, to help prevent the radiation from leaking out of the vials 172, 174, and getting into the ambient atmosphere around the cooler 10. It has been found by the applicant that because of the low radiation output of beta emitters such Yttrium, a polycarbonate material will work well to serve as the vial shields 182, 184.

One benefit of the use of a polycarbonate material is that it is transparent, which enables the user to monitor the level of material within the first and second vials 172, 174. Nonetheless, it is envisioned that other materials may be used, or may be necessary to use, if different types of radioactive materials are used, and which require different levels of shielding to prevent the leakage of dangerous radioactive emissions.

First 196, second 194, third 195, and fourth 197 medical material applicators which are here shown as syringes 196, 194, 195, and 197 are provided with the chillers, 10, 410 shown in the figures. The medical material applicators 196, 194, 195, and 197 are provided for applying the medical materials to the patient such as by implanting radioactive materials in tissue to help eradicate cancer cells in a patient.

Nonetheless, it will be appreciated that other models, sizes and configurations can exist within the scope and spirit of the invention. For example, a chiller device 10 may be employed that is capable of chilling a single syringe 190, or multiple syringes, such as 6, 8 or 12 syringes 190 that are in excess the present model shown in the drawings. In this regard, the device 10 can be scaled to increase or decrease the number and size of syringes 190 and vials 172, 174 that it is capable of accommodating different needs.

Additionally, the Vial receiver can be scaled to accommodate vials of different sizes. For example, the Chiller 10 can be scaled to hold 10 ml vials, 20 ml vials or even larger or smaller vials. device can be called to hold.

Further, the power of the Peltier chiller cooling component 78, 94 and its associated heat sinks can vary from what is shown, such as by employing a Peltier Chiller component (e.g.,78) that has, for example, four times the chilling capacity of the 48 W chillers shown in the drawings. If this higher capacity Peltier Cooler is used, the Peltier chilling can be accomplished with a single chiller and not the dual chillers 78, 94 shown in the drawings, since the aluminum block is an efficient thermal conductor. With a single chilling component, the device 10 can be reconfigured so that all the vials and the syringes are placed on top of the single Peltier cooler unit.

The upper casing member 16 includes a plurality of syringe receivers including a first syringe receiver 192 for receiving the first syringe 190; a second a syringe receiver 193 for receiving a second syringe 194, a third syringe receiver 196 for receiving a third syringe 195, and a fourth syringe receiver 198 for receiving a fourth syringe 197.

Each of the syringe receivers 192, 193, 196, 198 are generally cylindrical in shape, and are sized and configured for receiving generally cylindrical syringes 190, 194, 195, 197. The syringe receivers 192, 193, 196, 198 are also configured so that they are disposed relatively higher on the front panel of the device 10, and lower on the back panel of the device, so that when the syringes 190, 194, 195, 197 are placed in their respective syringe receivers 192, 193, 196, 198, the syringes are tilted at an oblique angle to the surface on which the casing rests, so that their distal, or needle ends are placed at a lower level than their proximal or plunger ends.

This angled placement helps to keep the material within the syringes 190, 194, 195, 197 collected near the proximal end of the barrel of the syringes. This places the material within the interior of the chiller, to help maintain the material within the syringes at the appropriate temperature.

Each of the syringe receivers 192, 193, 196, 198 has an open proximal and 202 into which the syringe can be inserted, and a closed distal end 204. A slotted thumbscrew receiving slot 208 is formed near the proximal end 202 of the syringe receivers, and is sized and configured for receiving a thumbscrew 210. The thumbscrew 210 can be rotated to move axially into and out of engagement with the syringe, to help maintain the syringe in position within the respective syringe receiver, to keep it from falling out and becoming damaged.

A cylindrical, round bottom shield 212 having a hollow interior, and a closed distal end is coupled to the distal portion of each of the syringe receivers 192, 193, 196, 198 to contain the distal end of the syringe therein. The round bottom shields 212 help to provide a shield against radioactive emissions.

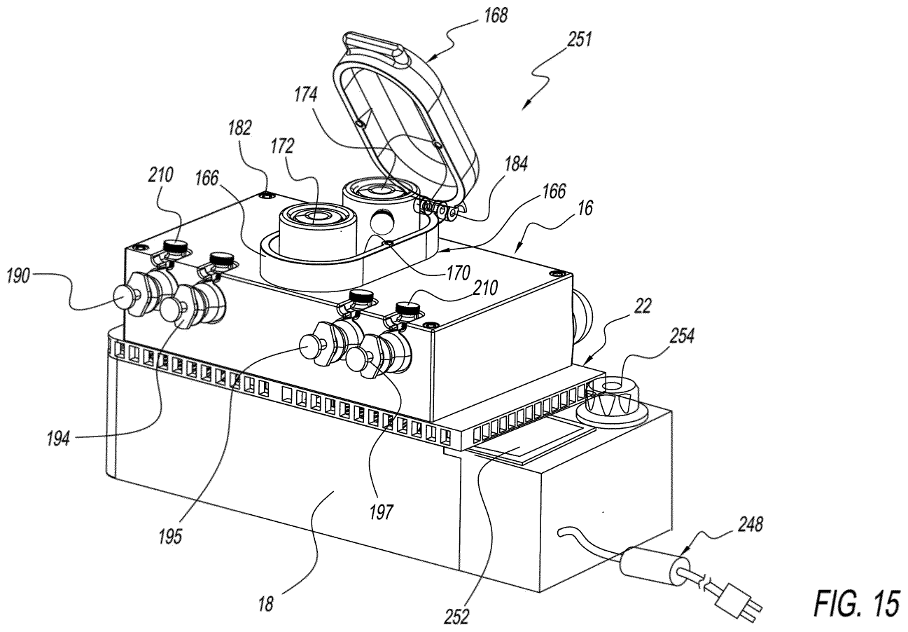

As best shown in FIG. 15, each of the syringes, e.g., 190 includes a proximal end 220 into which a plunger 222 is inserted, a central body or barrel portion 224, and a distally disposed needle portion 226. A needle cover 228 is placed over the distal needle portion 226, to prevent damage to the needle portion 226, and also to prevent the user from being inadvertently stuck by the needle of the distal needle portion 226.

An applicator receiving radiation shield 230 is provided for being received within each of the syringe receivers 192, 193, 196, 198, and is sized and configured for receiving the syringes 190, 194, 195, 197 within the hollow interior receiving cavity 238 of its own applicator receiving radiation shield 230.

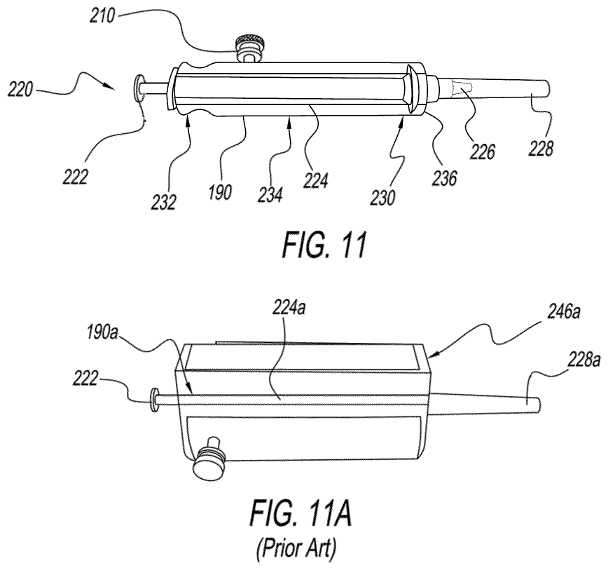

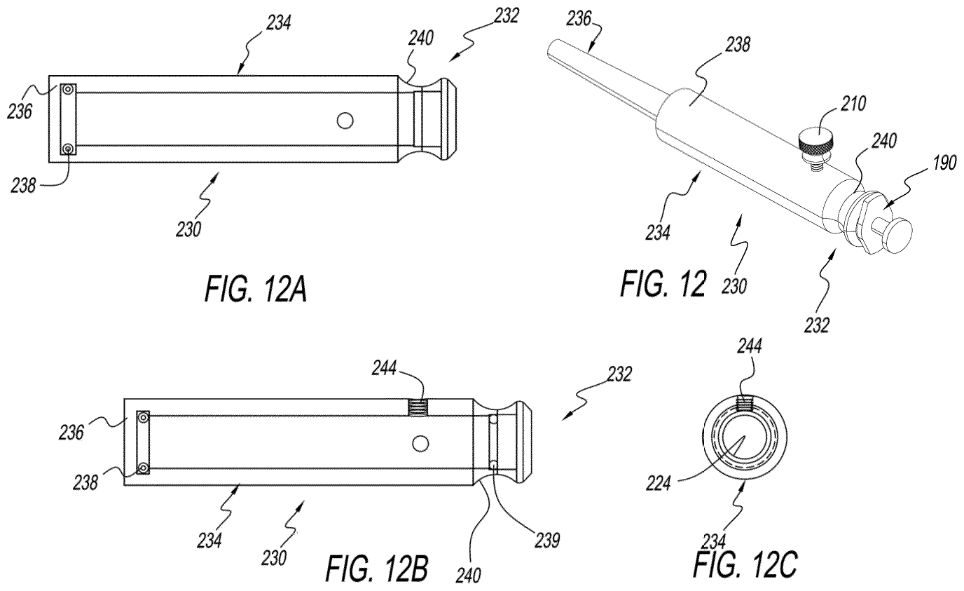

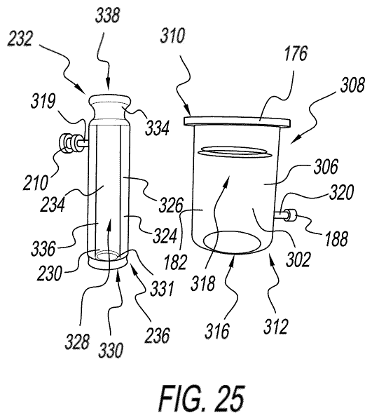

Referring now to FIGS. 11, 11A, 12A-12C, 25 and 26, an exemplary radiation attenuating applicator shield 230 will now be discussed.

The radiation attenuating applicator shield 230 is configured to surround and encase the syringe 190 and serves to attenuate radiation which is emitted from the radioactive material housed in the syringe 190. The radiation attenuating shield 230 will be referred to hereinafter as syringe shield 230. The syringe shield 230 is configured to reduce the amount of radiation a user is exposed to as they inject the target (e.g., patient) with the syringe 190, e.g., during a course of treatment.

The syringe shield 230 is depicted taking the form of a substantially hollow cylinder 326 which extends between a proximal end 232 and a distal end 236. The syringe shield 230 has a central body portion 234, which is defined by the outer wall 336. The central body portion 234 is configured to house and surround a central body of the syringe 190 therein. In this manner, the radiation emitted from the radioactive material housed within the central body of the syringe 190 will pass through the outer wall 336 of the syringe shield 230 prior to contacting a user.

The outer wall 336 of the syringe shield 230 is manufactured of a radiation attenuating material 324. In this manner, the syringe shield 230 will reduce the intensity (e.g., the level) of radiation to which the user is exposed. The outer wall 336 includes a thickness 340. As will be appreciated to a person of skill, the specific material 324 utilized as well as the thickness 340 of the material will impact the reduction (e.g., attenuation) in radiation provided by the syringe shield 230.

The radiation attenuating material 324 can take the form of polycarbonate. The Applicants found that the use of a polycarbonate material 324 provides suitable reduction/shielding capabilities for Beta radiation waves, which are the type of waves commonly emitted by the exemplary Yttrium radioactive materials often used in connection with the present invention. Advantageously, the poly carbonate 324 syringe shield 230 can also be transparent to thereby enable a user to easily view the syringe and markings through the syringe shield 230.

However, the syringe shield 230 may also be formed from a variety of radiation attenuating polymers, including PVC, acrylic, or the like, and/or can be formed of various other radiation attenuating materials. The material selected should be one that works well with the specific type of radiation emitted as well as the desired reduction in radiation. The syringe shield 230 can be scaled to house syringes 190 of various sizes and/or to house radioactive materials of varying intensities.

In an assembled configuration, with the syringe 190 properly located within the syringe shield 230, the needle portion 226 of the syringe 190 extends outwardly from an opening 330, toward the distal end 326; and the plunger 222 of the syringe 190 and extends outwardly from opening 338 which is located toward the proximal end 232.

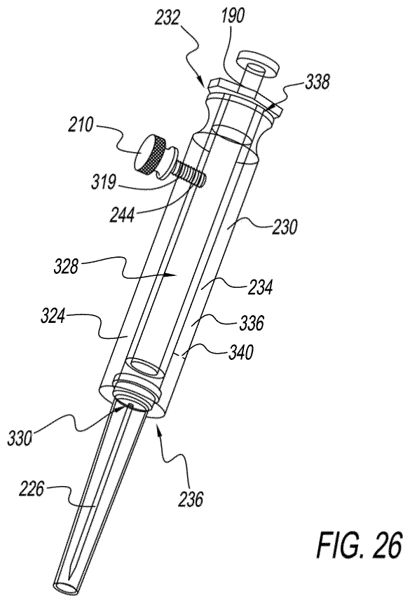

The syringe 190 is retained within the syringe shield 230 via a locking mechanism 210. As is best illustrated in FIG. 26, the thumb screw locking mechanism 210 includes a threaded rod 319 which extends through a threaded aperture 244 located in the outer wall 336.

When the locking mechanism 210 is screwed into the aperture 244, a distal end (not shown) of the threaded rod 319 will contact the syringe 190 to retain the syringe 190 within the syringe shield 230, which prevents longitudinal movement of the syringe 190 within the syringe shield 230.

An interior of the distal end 236 of the syringe shield 230 can include a reduced diameter portion or a stop to contact a distal end of the syringe 190. Preferably, an O-ring 331 is located at an internal surface of the syringe shield 230, toward the opening 330 at the distal end 236. The O-ring 331 is configured to snugly engage the syringe 190 to prevent movement of the syringe 190 within the syringe shield 230.

To insert a syringe 190 within the syringe shield 230, the needle portion 226 of the syringe 190 is inserted through the proximal opening 338. The needle portion 226 is passed through the hollow interior cavity 328 and is extended through the distal opening 330. With the needle portion 226 extending outwardly from the distal opening 330, the central portion of the syringe 190 is encompassed by the outer wall 336, and the locking mechanism 210 can be secured.

The syringe shield 230 is depicted as including a grasping portion 334, located toward the proximal end. The grasping portion 334 is configured to provide a location for a user to grasp the syringe shield 230. For example, a user may grasp the grasping portion 334 between their index and middle fingers as they operate the plunger 222 to draw radioactive material into the syringe 190 and/or to inject the radioactive material from the syringe 190.

Briefly turning to FIG. 14, construction of the syringe shield 230 from polycarbonate material 324 has been discovered to provide the syringe shield 230 with a relatively thin profile relative to syringe shields of the prior art, even though the thickness 340 of the material 324 of the outer wall 336 is sufficiently thick to provide the desired reduction in radiation. This relatively thin profile created by the reduced outer diameter of the syringe shield 230 (relative that of the prior art) is believed to make the syringe shield 230 less cumbersome and easier to utilize.

As best shown in FIG. 15, an electrical power source is provided for the invention. The electrical power source 248 preferably plugs into a standard 110 V AC outlet, and converts the power to a 12 V DC power output. The electrical power source can be a standard electrical power source, or can be one that includes a controller 249 for controlling the operation of the device, such as whether it is turned on or off. The controller 249 can also control the operations of the Peltier coolers 78, 94 to ensure that they cool the material to the proper temperature range, and maintain the temperature at that proper range. Alternately, a separate controller (not shown) can be provided on the device 10. As with most such electrical power sources, an outlet is provided on the device 10 into which the power source 248 plugs.

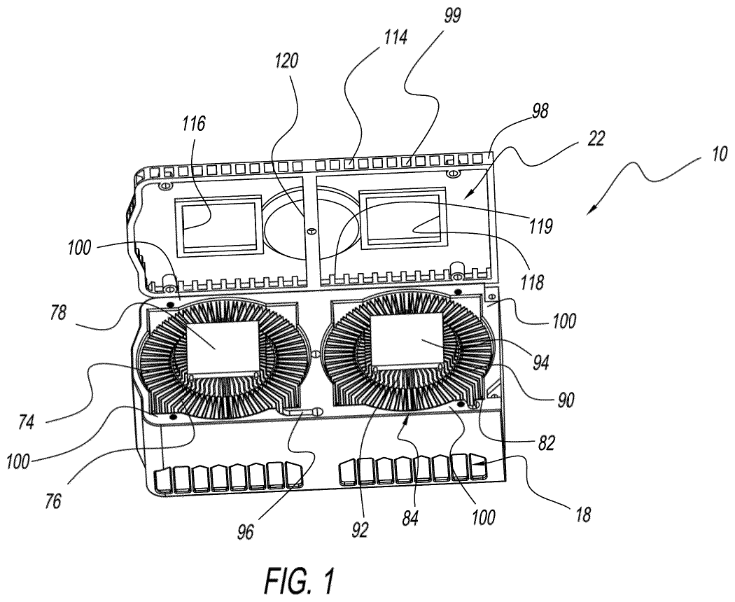

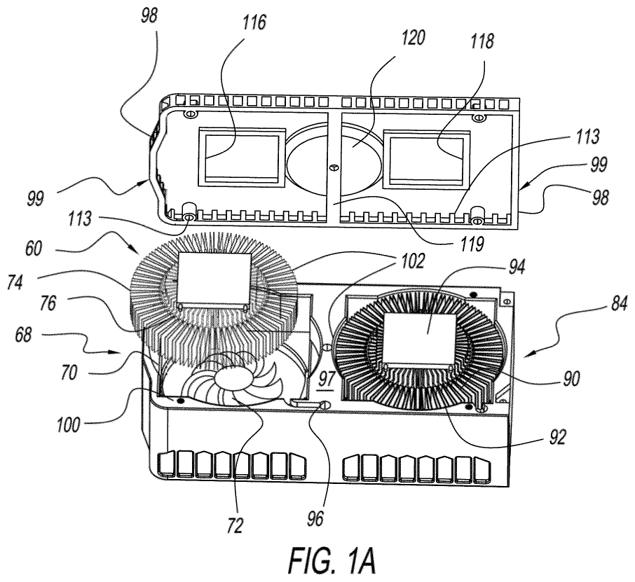

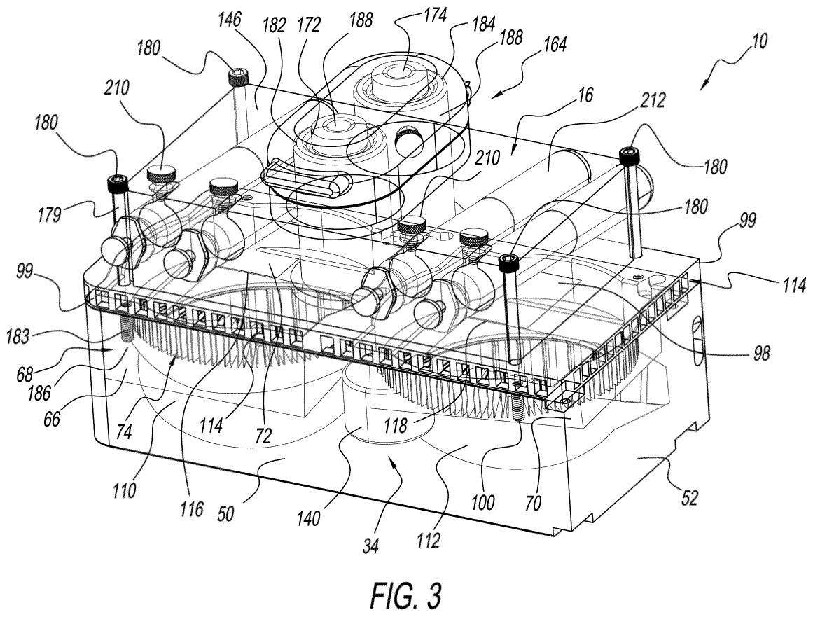

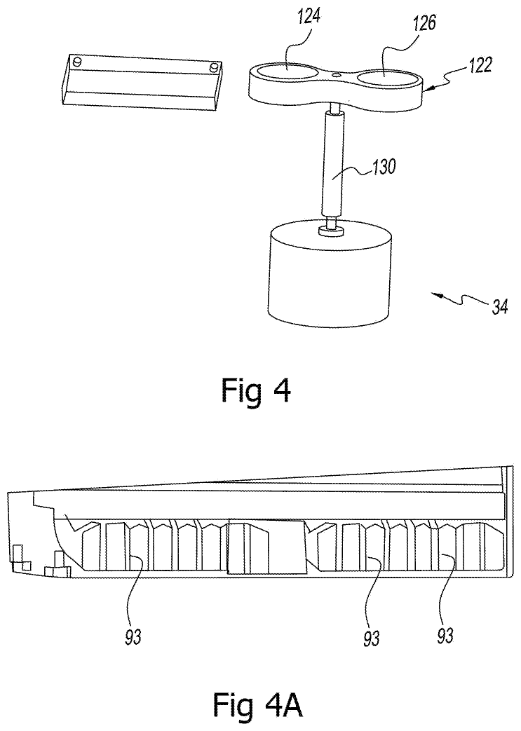

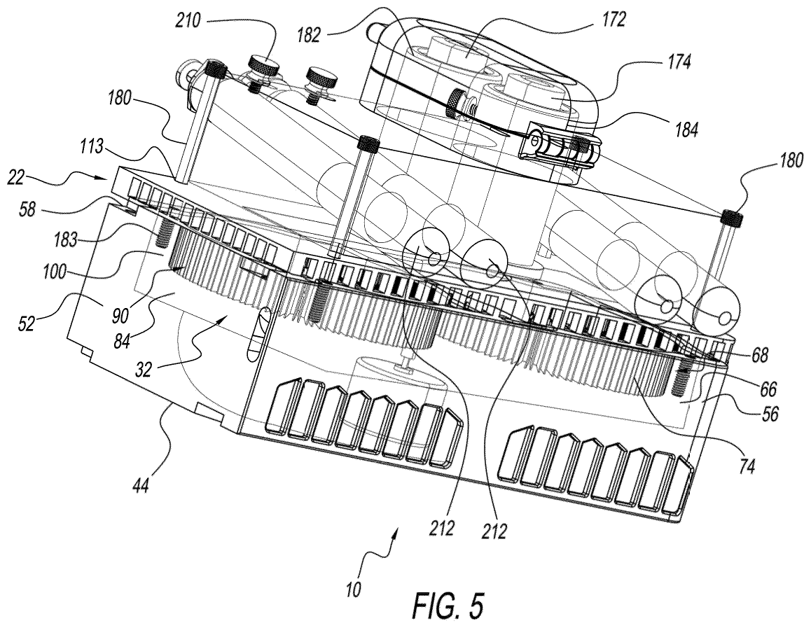

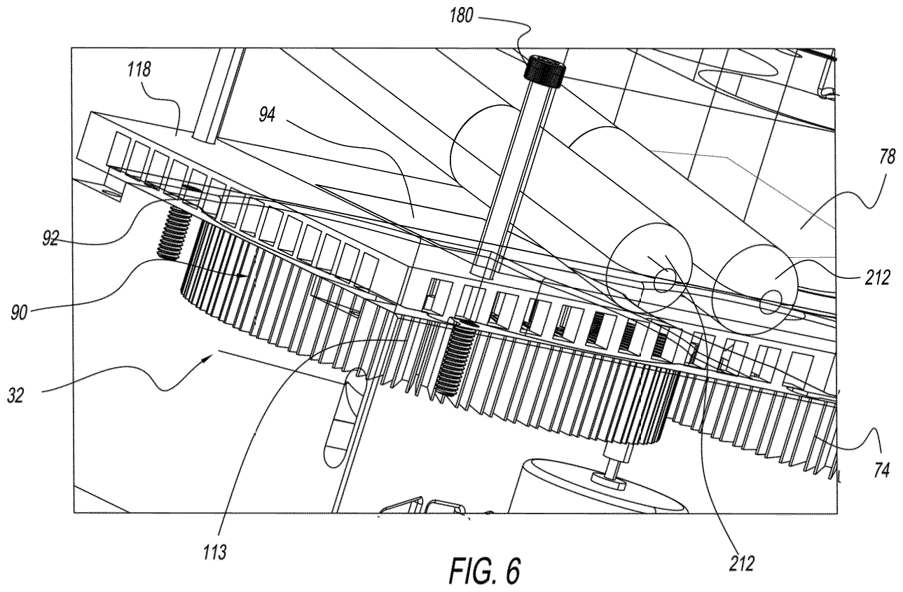

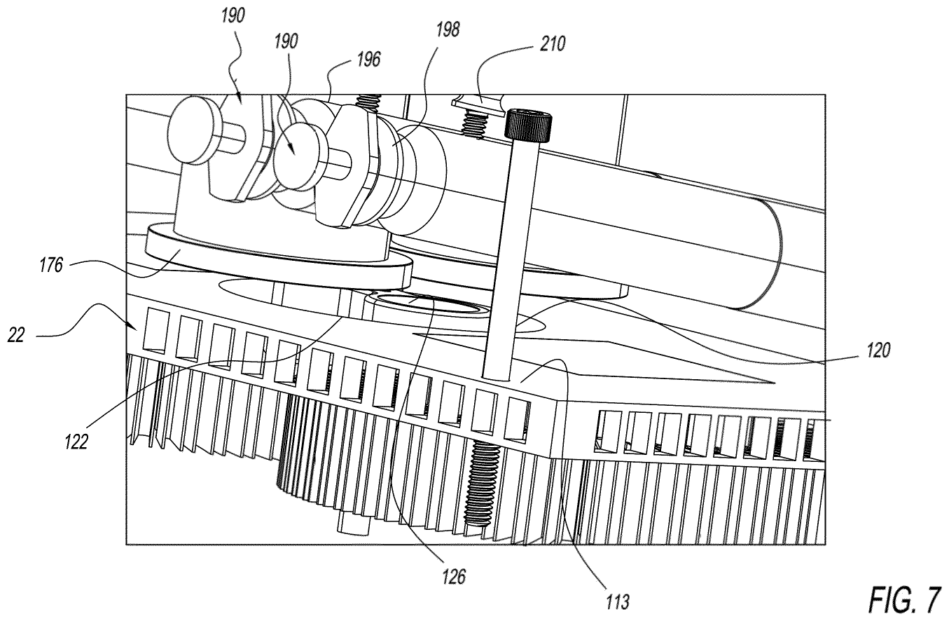

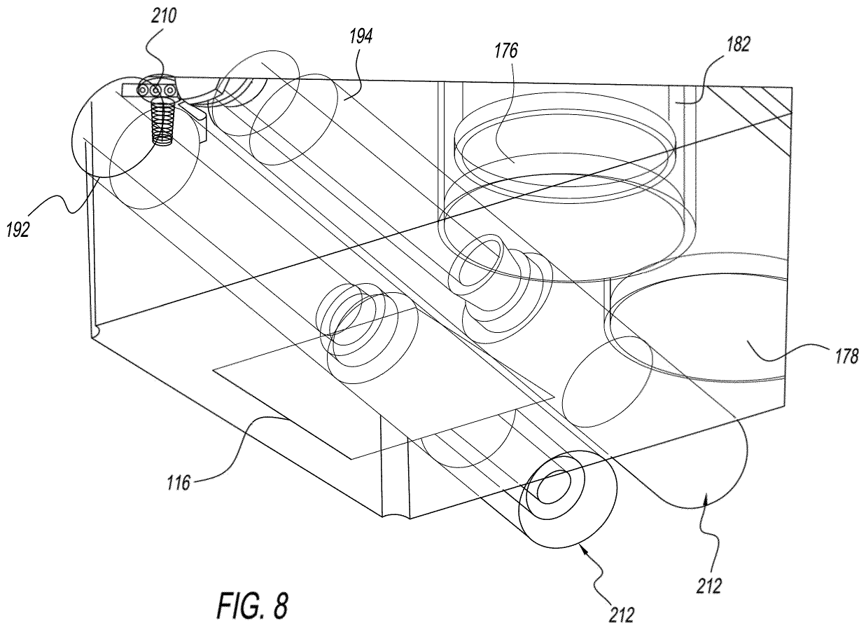

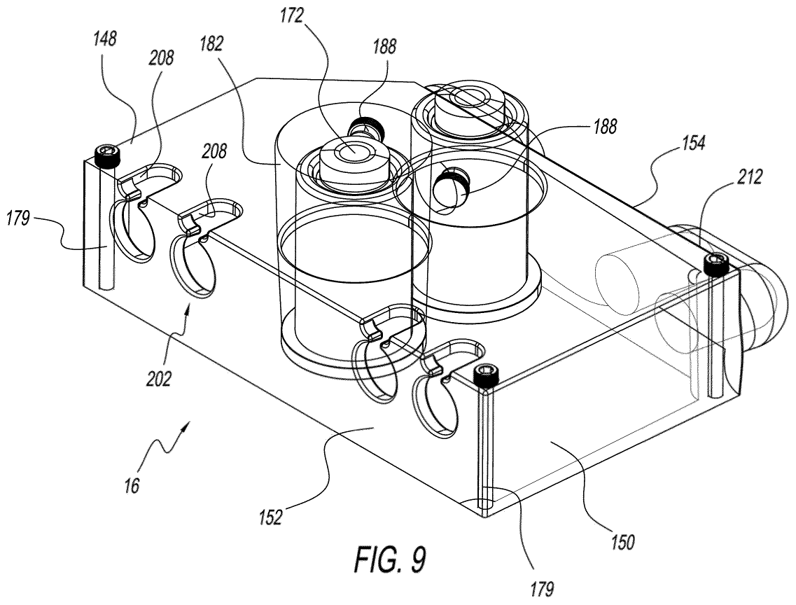

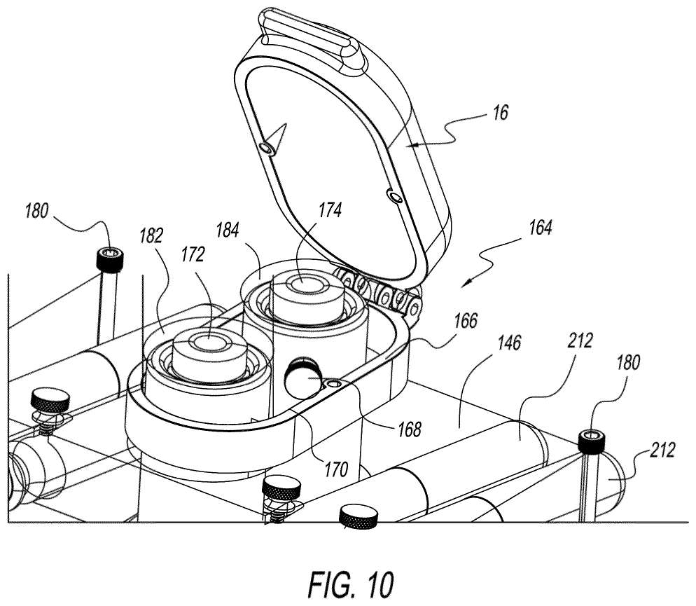

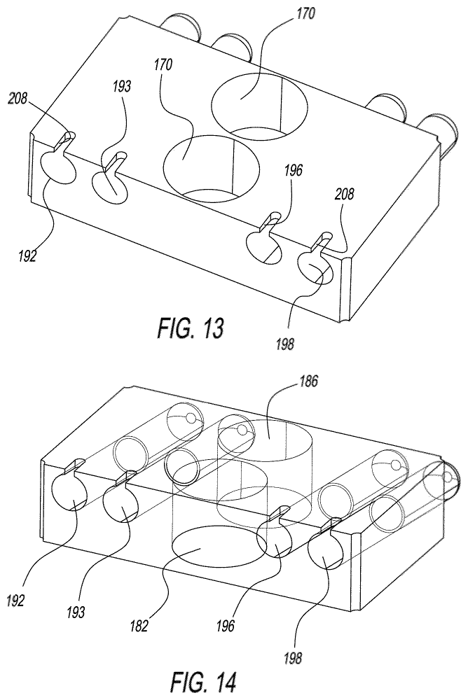

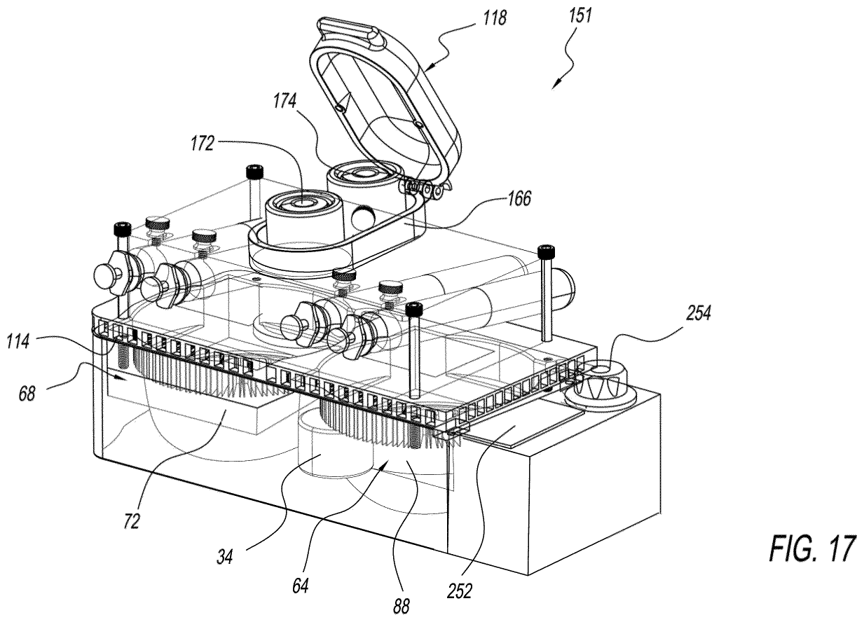

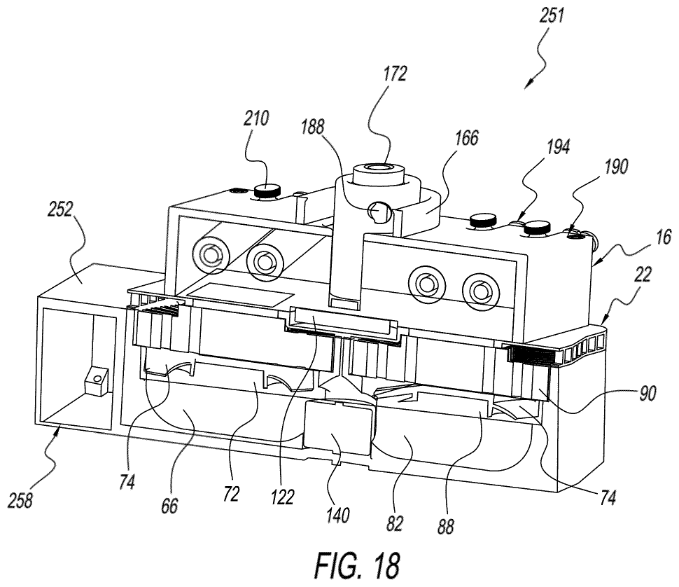

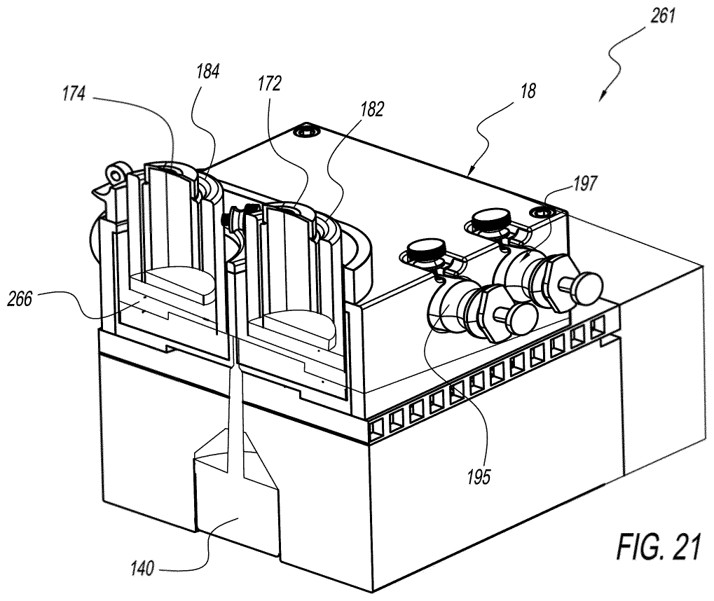

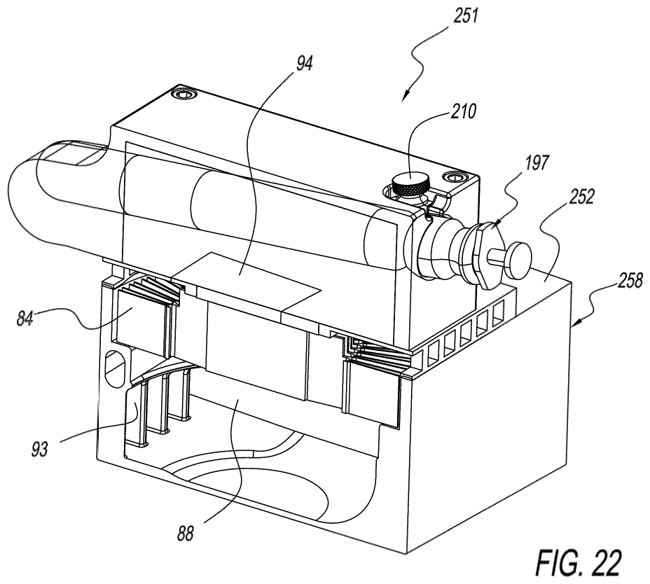

An alternate embodiment chiller device 251 is shown in FIGS. 13-22. The alternate embodiment device 251 is generally similar to the primary chiller device 10 described above, and includes similar components that are assigned numbers that are similar to their corresponding parts in the chiller 10. Primarily, the alternate embodiment chiller device 251 represents an improved version of the chiller device 10 shown in FIGS. 1-16.

The following components are notably changed in the alternate embodiment device 251. The first is the display containing controller 252, which operates the device. The controller 252 includes a display that displays the current parameters of the device, such as whether it is on or off, and the temperature of one or more interior portions of the device which should be reasonably close to the temperature of the materials contained within the syringes. Additionally, the display 252 may display the speed of rotation of the magnetic stirrer 34.

A primary purpose of the controller 252 is to control the temperature of the chiller 251, to thereby maintain the temperature of the medical materials at the proper temperature, or at least within the appropriate temperature range. Sensors (not shown) are in communication with, or are part of the controller 252, so that the controller 252 will know the real-time temperature of the aluminum block 266, or preferably, the shields for the test tubes.

A magnetic stirrer speed control knob 254 is disposed adjacent to the display containing controller 252. Both the magnetic stir speed control knob 254 and the display containing controller 252 are coupled to a box like controller housing 258 which is coupled to the side of the base casing member 16.

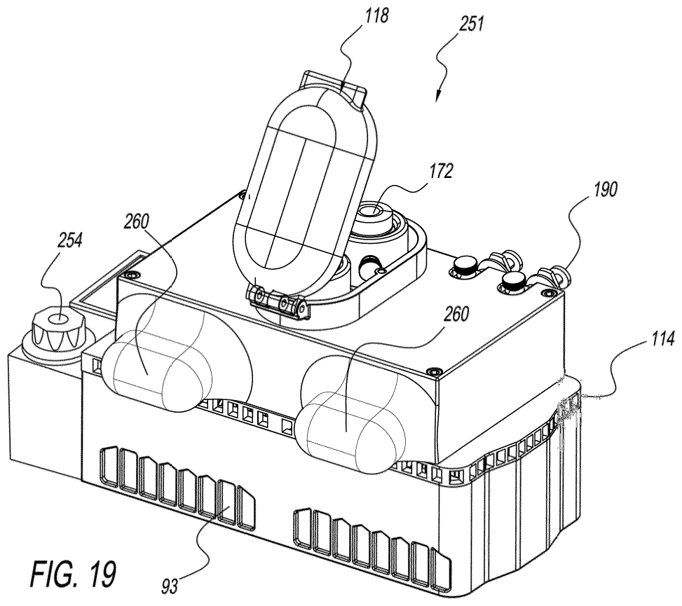

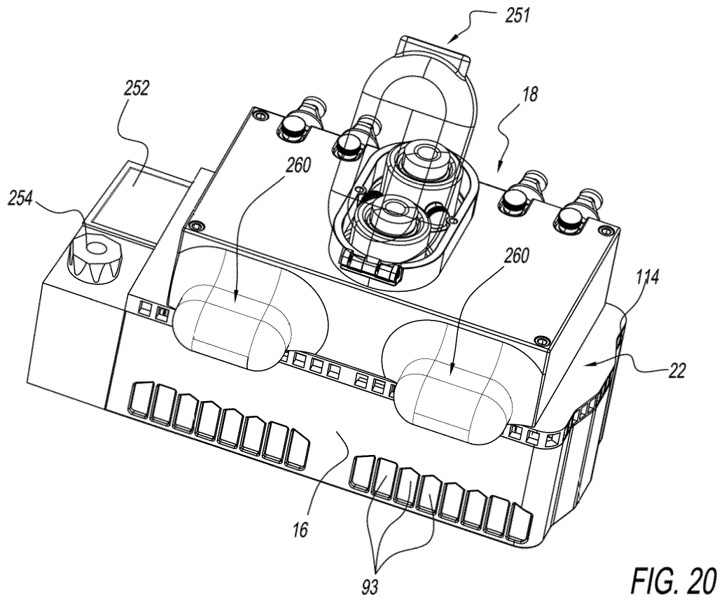

As shown in FIGS. 19 and 20, an alternate embodiment syringe bottom receiving shield 260 is shown, that includes enough interior space to receive the distal ends of two of the test tubes, rather than the single test tube capacity receiver shown in the chiller 10 of FIGS. 1-12.

Turning now to FIGS. 13 and 14, an aluminum block chiller 266 is shown. The aluminum block chiller 266 is sized and positioned to fit within the interior of the upper casing member 18. The aluminum block chiller 266 serves as a heat conductor, to exchange the heat from the Peltier cooler 78, 94, and place it adjacent to the vial housing 170, and test tube receivers 192, 193, 196, 198 to better chill the test tubes contained therein. Preferably, the chiller block 266 is made from an aluminum member, which is lightweight, and a very efficient heat conductor.

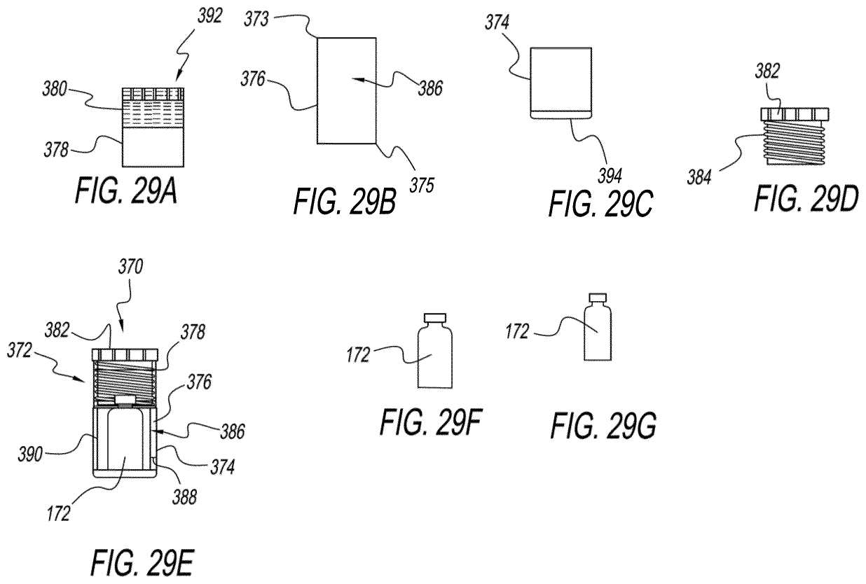

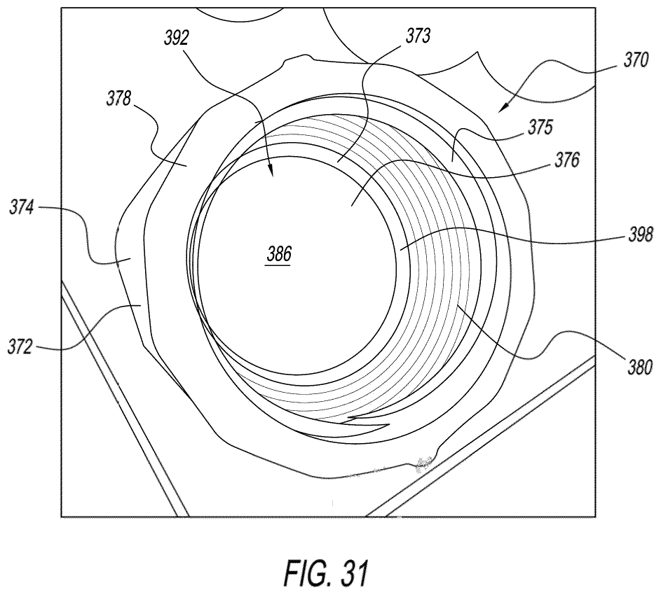

Referring now to FIGS. 29 and 31, an exemplary vial shield assembly 182 will now be described in more detail. The vial shield assembly 182 serves as a radiation shield for attenuating radiation to reduce exposure from the radioactive material housed within the vial 172. The vial shield assembly 182 extends between a closed lower portion 310, defined by a first vial receiving platform 176, and an upper portion 312. An outer wall 302 extends upwardly from the platform 176 to the upper portion 312.

A vial receiving cavity 318 is defined as being internal to the outer wall 302 above the base 176. The vial receiving cavity 318 includes a sufficiently large internal diameter 307 to house a vial 172 therein. The vial receiving cavity 318 is accessible via an opening 316 located at the upper portion 312.

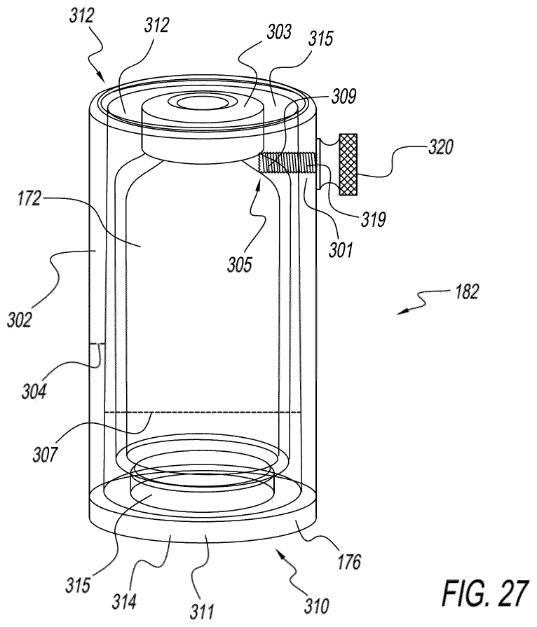

The vial shield assembly 182 (FIG. 27) can have a cylindrical shape 308 with a substantially hollow interior. The outer wall 302 is shown as having a height which is approximately equal to or greater than a height of the vial 172, which is depicted as being approximately equal to the vial cap 303.

As is best illustrated in FIG. 27, the outer wall 302 of the vial shield assembly container 182 surrounds the vial 303. The outer wall 302 is constructed of a radiation attenuating material such as polycarbonate. In this manner, radiation emitted by the radioactive material within the vial 172 will be attenuated by the outer wall 302.

The application found that using polycarbonate material 306 provides suitable radiation shielding for Beta radiation waves and advantageously is transparent. However, the vial shield assembly 182 can alternately be formed from a variety of radiation attenuating polymers, including PVC, acrylic, or the like, and/or can be formed of various other radiation attenuating materials. The material chosen is selected based upon the specific type of radiation emitted as well as the desired reduction in radiation.

The outer wall 302 has a thickness 304 which will impact the reduction in radiation. In general, the degree of shielding is proportional to the thickness of the outer wall 302.

The lower portion 310 of the vial shield assembly 182 includes base 176 that is configured to promote heat transfer between the vial 172 and the Peltier cooling elements 78 and 94 when the vial shield assembly 182 is disposed in the chiller 10. The base 176 is constructed of a heat conducting material 311, such as aluminum. However, the base 176 can be constructed from a variety of alternate heat conducting materials.

The base 176 is depicted as being a stepped base 176 that includes a lower base 314 portion and a reduced diameter upward extension 315 portion. A lower surface of the outer wall 302 rests atop the lower base 314. The upward extension portion 315 extends upwardly, and is disposed internally of the outer wall 302.

A lower portion of the vial 172 rests upon the upward extension 315. The lower base 314 and the upward extension 315 can take the form of flattened cylinders, being almost coin-like in shape.

The vial 172 is retained within the vial shield assembly 182 with locking mechanism 188. The locking mechanism 188 includes a threaded rod 320 which threading engages a threaded aperture 301 within the outer wall 302. When a user screws the threaded rod 320 inwardly, a distal end 309 of the threaded rod 319 with a thumb screw head 320 extends inwardly into the receiving cavity 318. When the locking mechanism 188 is in a locked position, the distal end 309 of the threaded rod 319 contacts the vial 172 at a location above a shoulder 305 of the vial 172, and retains the vial 172 within the interior cavity 318.

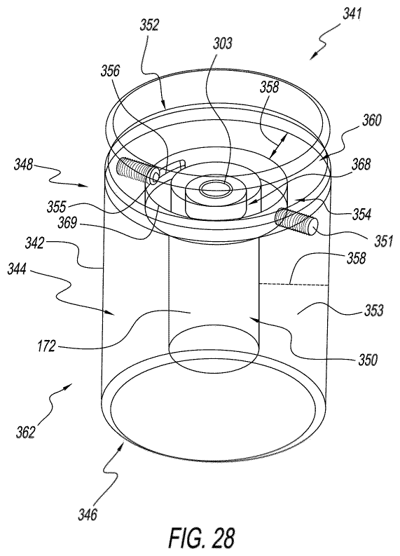

Referring now to FIG. 28, an exemplary radiation attenuating vial shipping container 341 is shown. This shipping container 341 includes a housing 342 which interiorly receives the vial 172 within a vial receiving cavity 350. A primary purpose of this housing 342 is to serve as a radiation shield for the radioactive material contained within the vial 172 during transit.

The housing 342 includes a base 344 and a lid 352. The base 344 extends between a closed lower portion 346 and an open upper portion 348. A vial receiving cavity 350 extends downwardly into the base 344, and is configured to receive and house a lower portion of the vial 172 therein.

A lower surface 369 of the lid 352 is depicted as including an upwardly extending chamber 368. This chamber 368 is configured to house an upper portion of the vial 172, such as the top 303 of the vial 172. When the lid 352 is secured on the base 344, the vial 172 is fully surrounded and encompassed by the container 340.

The shipping container 340 can include a locking mechanism 354. The locking mechanism 354 is configured to maintain the lid 352 securely on the base 344 during transit. As is illustrated, the locking mechanism 354 can take the form of a locking ball plunger which includes a threaded rod 351 with a spring-loaded plunger ball 355 extending therefrom.

The threaded rod 351 extends inwardly from the wall 353 of the base 344. A lower portion of the lid 369 includes a grooved channel 356 which is configured to cooperate with the spring-loaded plunger ball 355 to receive the plunger ball 355.

The insertion of the base 344 coupled plunger ball 355 into the channel 356 on the lid 352 locks the lid 352 securely to the base 344. Alternately, the lid 352 can securely be retained with the base 344 through various other mechanisms such as threaded, twist lock, snap-lock, bayonet mount, etc.

The lid 352 and the base 344 are constructed of a material 364 with radiation attenuating properties. The shipping container 340 is depicted as being formed of an acrylic 364. However, a variety of materials with suitable radiation attenuating properties may also be utilized.

Examples of such materials include PVC, polycarbonate, or other radiation attenuating materials. The base 344 and the lid 352 can be formed of similar or different materials, depending upon the desired properties of the finished container 340.

The base 344 should include a sufficiently great wall thickness 358 to provide the desired level of attenuation. As has been discussed, the thickness 358 required to ensure that a user is not over exposed to radiation will depend upon the properties of the specific radioactive material contained within the vial 172. These properties include its intensity of radiation and wavelength, as well as the material 364 from which the shipping container 340 is constructed. The thickness of the lid 352 as well as the thickness of the lower portion 346 should also be considered when determining the attenuation of the shipping container 340.

The shipping container 340 is depicted as including a substantially cylindrical exterior form 362. The lid 352 can include a recessed finger-engageable groove 360 to provide for ease of manipulation by a user.

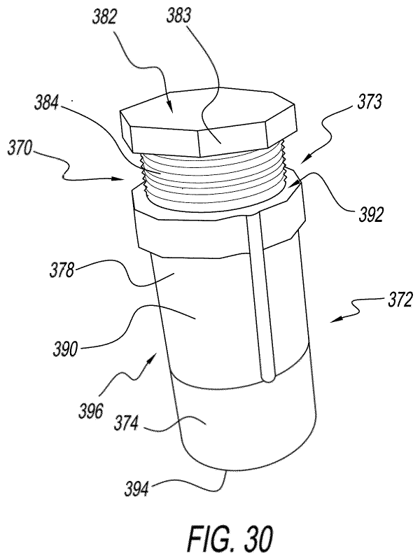

Referring now to FIGS. 29A-32 a further form of a radiation attenuating shipping container 370 will now be described. Similar to shipping container 341, the shipping container 370 serves as a radiation shield for attenuating the radiation released from the radioactive material within a vial 172, and also as a vessel for housing for the vial 172.

The shipping container 370 includes a base 372 and a lid 382. The base defines an interior cavity 386, that is configured to interiorly receive the vial 172 therein. The base 372 includes an upper portion 378 and a lower portion 374.

The base 372 includes a double-walled design. The base 372 includes an interior hollow liner 376 which extends between a first end 373 and a second end 375. The second end 375 of the liner 376 extends into the lower portion 374 and is affixed thereto. The first end 373 of the liner extends into the upper portion 374 and is affixed thereto. The hollow liner 376 defines the interior cavity 386 in which the vial 172 is disposed.

The lower portion 374 includes a closed, substantially planar bottom 394. An opening 392 is located in the upper portion 378. The opening 392 provides access to the interior cavity 386 when the threaded lid 382 is removed from its engagement with the female threaded opening 392 of the base 372 of container 370 therefrom.

When the lid 382 is engaged with the base 372, the shipping container 370 contains and encompasses the vial 172. In this manner, radiation that is emitted from the radioactive material within the vial 172 will need to travel through the walls of container 370 prior to reaching a human user.

The lid 382 includes a plurality of external male threads 384. These male threads 384 cooperate with and engage with female threads 380 which are formed on the radially inwardly facing interior wall to the upper portion 378, to selectively open and close the container 370. The lid 382 may include a hexagonally shaped top portion 383 to facilitate gripping and tuning by a hand or with a wrench.

The shipping container 370 is depicted as being constructed of PVC 180. However, the use of other radiation attenuating materials are contemplated herein. The shipping container 370 is depicted as having a substantially cylindrical form 396.

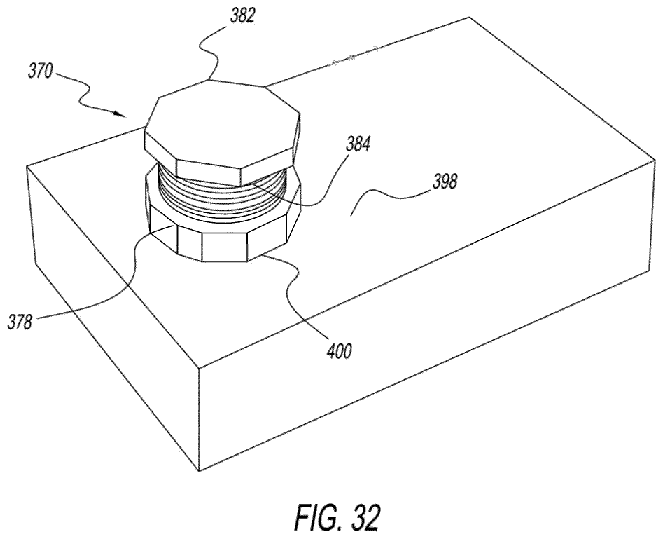

FIG. 32 depicts the shipping container 370 in a closed and ready to ship configuration. In this configuration, the vial 172 has been placed in the interior cavity 386 and the lid 382 has been screwed into the base 372 to thereby enclose the interior cavity 386 and encapsulate the vial 172 therein. The shipping container 370 may be inserted into a support block 398 to securely retain the container 370 during shipping.

This support block 398 is depicted as a Styrofoam block having an opening 400 therethrough that is sized and configured for snugly interiorly receiving the container 370. The lower portion 374 of the container 370 is inserted into and closely received by the opening 400. The support block 398, with the container 370 inserted therein, may then be packed and shipped in a common box or other external shipping container (not shown).

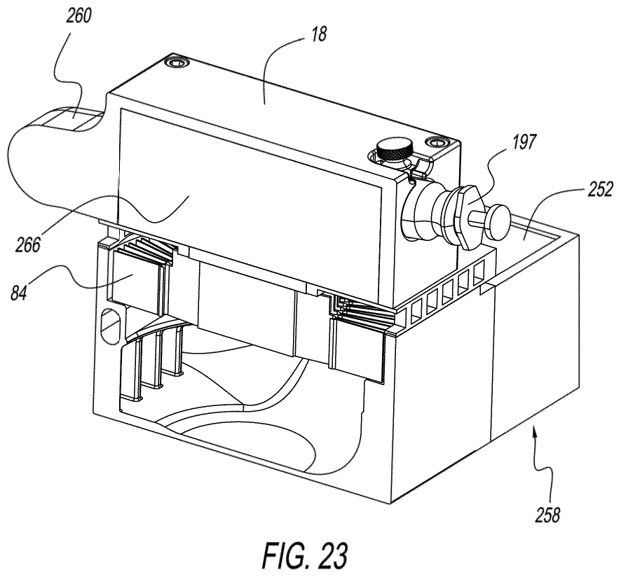

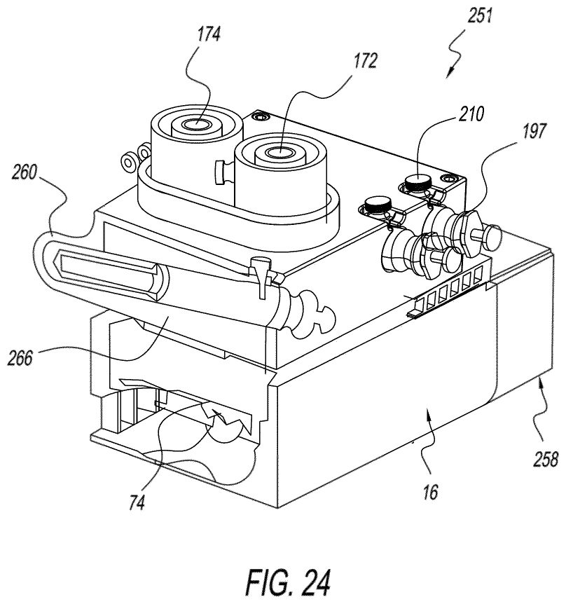

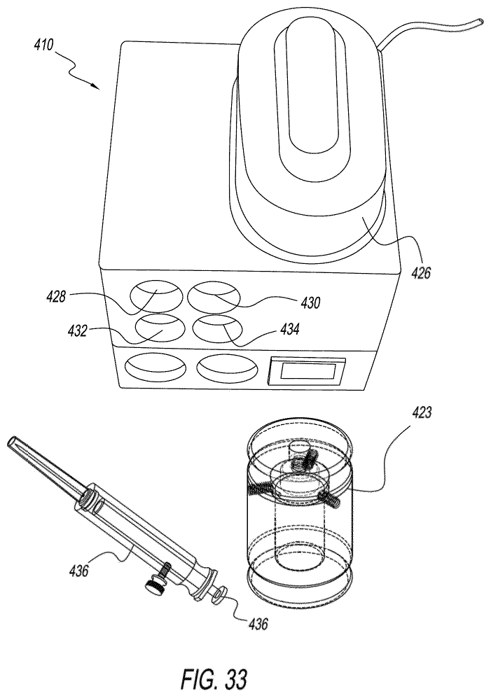

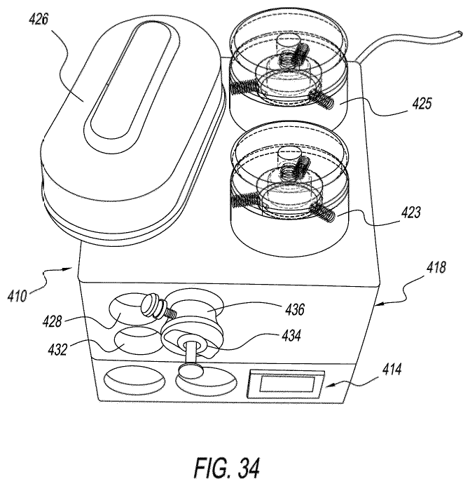

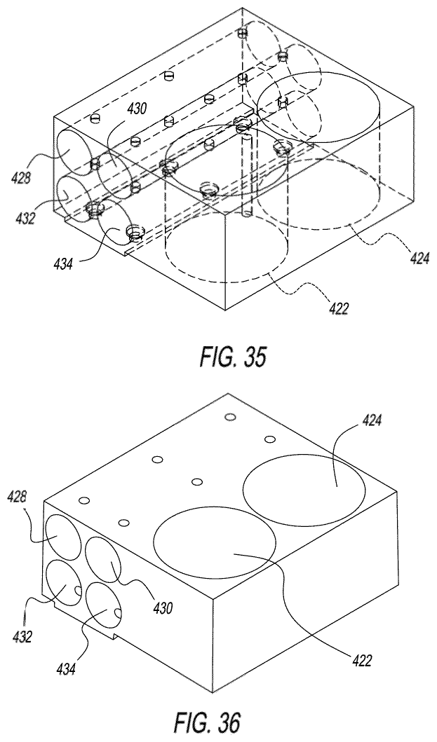

Turning now to FIGS. 33-36, another alternate embodiment chiller 410 as shown. Chiller 410 generally includes the same functional components as the chillers 10 discussed above, but arranges those components in a manner different than the manner in which the components are arranged in the chillers discussed above in connection with FIGS. 1-32.

Chiller 410 includes a first casing member 414, a second casing member (not shown) to which the thermoelectric cooler is mounted (not shown), and a third casing member 418.

The third casing member may comprise an aluminum block, into which the various features are drilled or machined. The third casing member 418 should include heat conductive materials, so that the materials contained by the third casing member 418 will be in thermal contact with the thermoelectric cooler (not shown) so as to maintain the materials at a temperature below ambient room temperature.

The chiller 410 also includes a first vial receiver 422 for receiving a first vial 423, and a second vial receiver 424 for receiving a second vial 425. The first and second vials 423, 425 are generally similar to those discussed above, and are preferably in contact with the magnetic stirrer (not shown) so that the medical materials contained therein can be stirred by the magnetic stirrer. A cap member 426 is provided to overlay the first and second vials 423, 425. The cap serves as an insulator for insulating the first and second vials 423, 425, and also serves as a radiation attenuator for insulating the ambient area around the chiller 410 from radioactive emissions that may be emitted by the medical materials within the first and second vials 423, 425. However, as discussed above, this radiation insulation feature serves primarily as a back up to the radioactive attenuation provided by the thickened polycarbonate material from which the first and second vial containers 423, 425 are made.

The third casing member 418 also includes a first applicator receiver 428, a second applicator receiver 430, a third applicator receiver 432, and a fourth applicator receiver 433. The applicator receivers are provided for interiorly receiving a syringe type applicator 434 that is encased within a radiation shield 436.

While the invention has been described in connection with what is presently considered to be the most practical and preferred embodiment, it is to be understood that the invention is not to be limited to the disclosed embodiments, but on the contrary, is intended to cover various modifications and equivalent arrangements included within the spirit and scope of the appended claims, which scope is to be accorded the broadest interpretation so as to encompass all such modifications and equivalent structures as permitted under the law. Accordingly, the scope of the present invention should be considered in terms of the following claims and is understood not to be limited to the details of structure and operation shown and described in the specification and drawings.

It should be understood that while the use of the word preferable, preferably, or preferred in the description above indicates that feature so described may be more desirable, it nonetheless may not be necessary and any embodiment lacking the same may be contemplated as within the scope of the invention, that scope being defined by the claims that follow.

In reading the claims it is intended that when words such as “a,” “an,” “at least one” and “at least a portion” are used, there is no intention to limit the claim to only one item unless specifically stated to the contrary in the claim. Further, when the language “at least a portion” and/or “a portion” is used the item may include a portion and/or the entire item unless specifically stated to the contrary.

The following is a listing of the numbered parts which have been disclosed and described in connection with the present invention.

| No. | Description |
|---|---|
| 10 | Medical Materials Chiller |
| 12 | Casing |
| 16 | Upper casing member |
| 18 | Lower casing member |
| 22 | Intermediate casing member |
| 26 | Upper Interior chamber |
| 28 | Lower interior chamber |
| 32 | Air handler assembly |
| 34 | Magnetic stirrer |
| 44 | Bottom panel |
| 48 | Four side panels |
| 50 | First short side panel |
| 52 | Second short side panel |
| 54 | First long side panel |
| 56 | Second long side panel |
| 58 | Upper edge surface |
| 66 | First heat sink receiving chamber |
| 68 | First active heat sink |
| 70 | Fan receiving shelf |
| 72 | Fan |
| 74 | First Finned aluminum first heat sink |
| 76 | Reduced diameter central portion |
| 78 | First thermoelectric (Peltier) cooler |
| 82 | Second heat sink receiving chamber |
| 84 | Second active heat sink |
| 86 | Fan receiving shelf |
| 88 | Second fan |
| 90 | Second Finned aluminum second |
| | heat sink |
| 92 | Reduced diameter central platform |
| 93 | Plurality of exhaust apertures |
| 94 | Second thermoelectric (Peltier) |
| | cooler |
| 96 | Upper edge |
| 97 | Transverse central member |
| 98 | Lower edge |
| 99 | Perimetral skirt |
| 100 | Bolt receiving apertures |
| 102 | Output shaft |
| 104 | Output shaft |
| 106 | Horizontally disposed panel |
| 110 | First intake chamber |
| 112 | Second intake chamber |
| 113 | Bolt receiving aperture |
| 114 | Intake apertures |
| 116 | First thermoelectric (Peltier) cooler |
| | receiving aperture |
| 118 | Second thermoelectric (Peltier) |
| | cooler receiving aperture |
| 119 | Transverse member |
| 120 | Central recessed portion |
| 122 | Magnet housing |
| 124 | First magnet member |
| 126 | A second magnet member |
| 128 | Thermoelectric cooler |
| 130 | Central aperture |
| 132 | Hot side |
| 133 | First ceramic substrate |
| 134 | Cold side |
| 135 | Second ceramic substrate |
| 136 | P-type semiconductor pellet |
| 138 | N-type semiconductor pellet |
| 139 | First (positive) lead |
| 140 | Motor |
| 141 | First copper contact |
| 142 | Second (negative) lead |
| 143 | Second copper contact |
| 146 | Top panel |
| 147 | Bottom panel |
| 148 | First short side panel |
| 150 | Second short end side panel |
| 152 | First long end side panel |
| 154 | Second long end side panel |
| 164 | Vial receiver |
| 166 | Collar portion |
| 168 | Hingedly coupled cap |
| 170 | Vial receiving chamber |
| 172 | First vial |
| 174 | Second vial |
| 176 | First coin-shaped vial receiving |
| | platform |
| 178 | Second coin-shaped vial receiving |
| | platform |
| 179 | Four bolt receiving passageways |
| 180 | Four threaded bolts |
| 183 | Threaded ends |
| 182 | First cylindrical polycarbonate vial |
| | receiver and shield |
| 184 | Second cylindrical polycarbonate |
| | vial receiver and shield |
| 186 | Threaded bolt receiving apertures |
| 188 | Vial thumbscrews |
| 190 | First medical material applicator |
| | (syringe) |
| 192 | First medical applicator (syringe) |
| | receiver |
| 193 | Second syringe receiver |
| 194 | Second syringe |
| 195 | Third syringe |
| 196 | Third syringe receiver |
| 197 | Fourth syringe |
| 198 | Fourth syringe receiver |
| 202 | Open proximal end |
| 204 | Closed distal end |
| 208 | Slotted thumbscrew receiving portion |
| 210 | Thumbscrew (locking mechanism) |
| 212 | Cylindrical round bottomed shield |
| 218 | Syringe |
| 220 | Proximal end |
| 222 | Plunger |
| 224 | Barrell portion |
| 226 | Distal needle portion |
| 228 | Needle cover |
| 230 | Radiation attenuating applicator |
| | shield |
| 232 | Proximal end |
| 234 | Central body portion |
| 236 | Diameter reduced distal end |
| 238 | O-ring |
| 239 | Second O-ring |
| 240 | Reduced diameter necked down |
| | portion |
| 244 | Threaded aperture |
| 246 | Prior art needle radiation shield |
| 248 | Electrical power source for |
| | converting 110 V AC to 12 |
| | Volt DC output |
| 250 | Passageway |
| 251 | Alternate embodiment chiller |
| 252 | Display containing controller |
| 254 | Magnetic stirrer speed control knob |
| 258 | Box like controller housing |
| 260 | Alternate embodiment syringe |
| | bottom shield |
| 266 | Aluminum block |
| 302 | Outer wall |
| 303 | Vial |
| 305 | Shoulder |
| 307 | Internal chamber |
| 309 | Cylindrical distal end |
| 310 | Closed lower portion |
| 312 | Upper portion |
| 314 | Lower base portion |
| 315 | Upward exterior opening |
| 318 | Vial receiving cavity |
| 319 | Threaded rod |
| 324 | Radiation attenuating poly carbonate |
| 326 | Hollow cylinder |
| 328 | Hollow interior cavity |
| 330 | Distal opening |
| 331 | O-ring |
| 332 | |
| 334 | Grasping portion |
| 336 | Outer wall |
| 338 | Proximal opening |
| 340 | Thickness |
| 341 | Shipping container |
| 342 | Housing |
| 344 | Base |
| 346 | Closed lower portion |
| 348 | Open upper portion |
| 350 | Vial receiving cavity |
| 351 | Threaded rod |
| 352 | Lid |
| 354 | Locking mechanism |
| 355 | Plunger |
| 358 | Wall thickness |
| 368 | Upwardly extending chamber |
| 369 | Lower surface |
| 370 | Alternate embodiment shipping |
| | container |
| 372 | Base |
| 373 | First end |
| 374 | Lower portion |
| 375 | Second end |
| 376 | Interior hollow liner |
| 378 | Upper portion of base |
| 382 | Lid |
| 383 | Hexagonally shaped top portion |
| 384 | Male threads |
| 386 | Interior cavity |
| 392 | Opening |
| 394 | Flattened planar bottom |
| 398 | Support block |
| 400 | Opening |
| 410 | Chiller |
| 412 | Casing |
| 414 | First casing member |
| 418 | Third casing member |
| 422 | First vial receiver |
| 423 | First vial |
| 424 | Second vial receiver |
| 425 | Second vial |
| 426 | Cap |
| 428 | First applicator receiver |
| 430 | Second applicator receiver |
| 432 | Fourth applicator receiver |
| 434 | Syringe type |
| 436 | Radiation shield |

What is claimed is:

1. A chiller for maintaining medical materials at a temperature below ambient room temperature, the chiller comprising:
a. a casing having an interior configured for receiving a plurality of components,
b. a thermoelectric cooler coupled to and supported by the casing, the thermoelectric cooler including a hot side and a cold side,
c. a fluid mover for moving fluid into thermal contact with the hot side to remove heat from the hot side,
d. a first vial receiver in thermal contact with the cold side of the thermoelectric cooler, the first vial receiver being configured for receiving a first vial having an interior configured for receiving medical material,
e. a second vial receiver in thermal contact with the cold side of the thermoelectric cooler, the second vial receiver being configured for receiving a second vial having an interior configured for receiving medical material; and
f. a stirrer for stirring the medical material in the first vial and/or the second vial.

2. The chiller of claim 1 wherein the first and second vials are configured for shipping and/or storage of the medical material.

3. The chiller of claim 2 wherein the first vial receiver comprises a plastic container having a metal bottom for facilitating heat transfer between an exterior and an interior of the container, and wherein the container is comprised of a radiation attenuating material.

4. The chiller of claim 2 wherein the first vial comprises at least two first vials, and wherein the stirrer is configured for stirring medical materials in the first and second vials.

5. The chiller of claim 1 further comprising at least one applicator configured for applying medical materials to a patient and the chiller includes at least one applicator receiver in thermal contact with the thermoelectric cooler for maintaining medical materials in the applicator below ambient temperature.

6. The chiller of claim 5 further comprising an applicator shield for interiorly receiving the applicator, and wherein the applicator comprises a body having an exterior surface and an interior surface for defining an interior needle engaging distal end, an open, plunger receiving proximal end, and a plunger insertable in the interior, wherein the applicator shield includes an annular groove configured to be engaged by the user's fingers for facilitating movement of the applicator shield and the applicator.

7. The chiller of claim 6 further comprising a locking mechanism engageable with the applicator and applicator receiver for fixedly positioning the applicator in the applicator receiver.

8. The chiller of claim 6 wherein the locking mechanism includes a slot formed at a proximal end of the applicator receiver and a thumb screw insertable in the slot and engageable with the applicator shield.

9. The chiller of claim 6 wherein the applicator receiver is coupled to the casing and comprises a tubular cavity having an open proximal end for receiving the applicator and a closed distal end, the proximal end of the tubular cavity being disposed at a higher position than the distal end to position an applicator placed therein at an oblique angle relative to a chiller resting surface.

10. The chiller of claim 1 further comprising an applicator receiver and an applicator configured for applying medical materials to a patient,
the applicator receiver comprising at least one tubular passageway configured for receiving the applicator within the passageway.

11. The chiller of claim 10 further comprising an applicator receiving radiation shield having a hollow interior for interiorly receiving the applicator, and an applicator locking mechanism engageable between the applicator radiation shield and the applicator, for maintaining the applicator within the hollow interior of the radiation shield.

12. The chiller of claim 11 wherein the applicator radiation shield includes an annular groove, and the locking mechanism comprises a threaded screw having an end receivable into the applicator radiation shield, and engageable with the applicator.

13. The chiller of claim 10 wherein the applicator receiver includes a thermally conductive support member coupled to the casing and in thermal contact with the thermoelectric cooler, the support member including at least two tubular passageways configured for receiving tubular shaped applicators having a proximal end and a distal end, the two tubular passageways including a relatively raised proximal end for receiving the proximal end of the tubular shaped applicators and a distal end for receiving the distal end of the tubular shaped applicators, to position the applicators at an oblique angle to a resting surface upon which the chiller rests.

14. The chiller of claim 1 wherein the casing includes a first casing member for housing the fluid mover, a second casing member to which the thermoelectric cooler is mounted, and a third casing member to which an applicator receiver is coupled, the applicator receiver being in thermal contact with the thermoelectric cooler.

15. The chiller of claim 1 wherein the casing includes a lower casing member, further comprising a heat sink in thermal contact with the hot side of the thermoelectric cooler, and
wherein the casing includes apertures for allowing cooling fluid to pass between ambient atmosphere and the interior of the casing and the heat sinks.

16. The chiller of claim 15 wherein the fluid mover comprises an air mover for drawing ambient air into the interior of the first casing member, moving the ambient air through the apertures and into the interior of the first casing member, moving the air past the heat sink to absorb heat from the heat sink and exhausting the heated air from the casing through the apertures.

17. The chiller of claim 16 further comprising a heat sink receiving chamber for encasing the heat sink.

18. A container for encasing a vial containing a radioactive material, the container being comprised of a radiation attenuating material having a bottom, a side wall including an exterior surface, an interior surface and a vial receiving interior cavity, the interior cavity having a top opening, and defined by the interior surface and a thickness defined by the distance between the interior surface and the exterior surface, and
a cap member placeable over the top opening, the cap member having a top surface, a bottom surface and a thickness defined by the distance between the top surface and the bottom surface,
wherein the thicknesses of the cap member and the side wall is sufficiently great so as to absorb sufficient radiation emitted by the radioactive medical materials, so that the radioactivity levels outside the container are at acceptably safe levels.

19. The container of claim 18 further comprising a locking mechanism for maintaining a second container within the container, and
wherein the container includes a metal bottom for facilitating cooling of the container, and the radioactive materials contained within the second container.
